(19) United States
(12) Patent Application Publication
(10) Pub. No.: US 2013/0143855 A1
(43) Pub. Date: Jun. 6, 2013

(54) METHOD TO ALLEVIATE DIABETIC END STAGE RENAL DISEASE

(71) Applicant: Bin-Xian Zhang, San Antonio, TX (US)

(72) Inventor: Bin-Xian Zhang, San Antonio, TX (US)

(73) Assignee: BOARD OF REGENTS OF THE UNIVERSITY OF TEXAS SYSTEM, Austin, TX (US)

(21) Appl. No.: 13/699,823

(22) PCT Filed: May 25, 2011
(86) PCT No.: PCT/US11/37866

§ 371 Date: Feb. 12, 2013

Related U.S. Application Data
(60) Provisional application No. 61/348,638, filed on May 26, 2010.

(51) Int. Cl. A61K 31/395

(52) U.S. Cl. CPC A61K 31/395
USPC 514/183

(57) ABSTRACT

A method of alleviating end-stage renal disease (ESRD), comprising administering an effective amount of a gel-danamycin derivative.

### BACKGROUND OF THE INVENTION

1. Field of the Invention

The invention generally relates to the treatment of kidney disease. More particularly, the invention generally relates to the treatment of kidney problems associated with kidney failure.

2. Description of the Relevant Art

Diabetes remains the leading cause of End Stage Renal Disease (ESRD) in the United States. Although a large number of diabetic mouse models have been identified and their kidney complications develop to varying degrees, none has exhibited all the functional and histopathological features of ESRD found in patients with T2DM. The db/db mouse (BKS.Cg-m+/+Lepr^{db}/J mouse, Jackson Laboratory) is a widely used model for T2DM and its characteristics of nephropathy have been extensively defined. Certain features of human diabetic nephropathy, such as renal hypertrophy, glomerular enlargement, albuminuria and mesangial matrix expansion, have been confirmed in db/db mice. However, other key features of diabetic ESRD including lack of progressive albuminuria, nodular glomerulosclerosis, tubular atrophy, and tubulointerstitial fibrosis, are not reliably mimicked in db/db mice. The most striking deficiency of all the available mouse models, including the db/db model, is that these animals do not develop severe renal functional insufficiency (e.g., >80% loss of GFR or urine output, as observed in human ESRD) or renal failure to serve as suitable models for diabetic ESRD research. Whether these models are resistant to ESRD development or require lengthy nurturing prior to developing ESRD is unclear. Regardless of the reasons behind this deficiency, a model that conveniently mimic the severe renal insufficiency of human ESRD is urgently needed for elucidating the pathogenesis of diabetic ESRD, and for developing innovative and effective strategies to prevent ESRD.

Although hyperglycemia per se is a critical factor for developing vascular complications in T2DM, a recent intervention study with a large patient cohort strictly controlling glycemia not only failed to significantly reduce the incidence of cardiovascular disease, but also increased mortality in T2DM patients compared with standard therapy. Similarly, strict glycemic control slows but does not prevent diabetic nephropathy. Factors other than hyperglycemia are clearly involved in the pathogenesis of diabetic complications; one putative factor is elevated serum concentrations of nonesterified fatty acids (NEFA). Excessive NEFA not only contribute to insulin resistance by various mechanisms but also cause mitochondrial defects. In insulin resistant and T2DM subjects, the normalization of mitochondrial morphology, oxidative capacity and mitochondrial density by diet and/or exercise indicates that the mitochondrial defects under these conditions are caused mainly by the imbalance between calorie intake and consumption. We have demonstrated that polyunsaturated fatty acids (PUFA), such as linoleic acids (LA; 18:2, n-6), depletes [Ca²⁺]ₘ by a mechanism of polyunsaturated fatty acid-induced mitochondrial Ca²⁺ efflux (PIMCE) with the peroxynitrite overproduction and increased protein nitrotyrosylation; heat shock protein 90β1 (hsp90β1) plays an essential role in mediating this process.

### SUMMARY OF THE INVENTION

Type 2 diabetes mellitus (T2DM) is the leading cause of end stage renal disease (ESRD). Lack of suitable animal models hampers the progress of basic and clinical research in diabetic ESRD. Based on challenge of db/db mice with high fat diet (HF), we established a mouse model that developed ESRD and mimicked the typical functional and histopathological features of renal failure in T2DM patients. Using this model, we demonstrated that the geldanamycin derivatives preserved kidney function and ameliorated glomerular and tubular damages. Geldanamycin derivatives also significantly extended survival of the animals and protected them from the high mortality associated with ESRD. In the kidney, HF enhanced 3-nitrotyrosine (3-NT) levels and the geldanamycin derivatives inhibited this effect. Treatment with geldanamycin derivatives may contribute to diminished 3-NT levels, improved kidney function and enhanced animal survival by downregulating mitochondrial hsp90β1 levels and attenuating mitochondrial Ca²⁺ ([Ca²⁻]ₘ) efflux. In one embodiment, the geldanamycin derivative 17-dimethylaminoethylamino-17-demethoxygeldanamycin (“17-DMAG”) may be used to treat ESRD.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts kidney functions of db/db mice during high fat diet (HF) challenge and 17-DMAG treatment. (FIG. 1A) Schematic of two phases of HF feeding and 17-DMAG treatment with the arrows indicating the scheduled kidney function assessments. During the first HF and subsequent regular diet (RD) feeding, the 24 h urinary albumin excretion (FIG. 1B) and urine output (FIG. 1C) were measured to assess kidney functions in db/db and db/+ mice. During the second phase of HF feeding with 17-DMAG treatment, the 24 h urinary albumin excretion (FIG. 1D) and urine output (FIG. 1E) were initially assessed for kidney functions; subsequently, serum creatinine (FIG. 1F) was measured when anuria or oliguria occurred. **P <0.01, compared with baseline assessed on day 0 with n=6-12 per group;

FIG. 2 depicts kidney histopathology of HF-fed db/db mice. Representative figures showing the photomicrographs of HE (FIGS. 2A-2F), PAS (FIGS. 2G, 2H), and Masson's trichrome stained sections from db/db-HF-S group (FIG. 2C, 2E, 2G, 2I) and db/db-HF-D group (FIGS. 2D, 2F, 2H, 2J) taken at 100× (FIGS. 2A-2F) or 200× (FIGS. 2G-2J) magnification. Image-based computer assisted analysis was performed to quantify tubular damage index (FIG. 2K), mesangial expansion (FIG. 2L), and interstitial collagen accumulation (FIG. 2M) from 6 animals per group;

FIG. 3 depicts the effect of 17-DMAG on survival rate of HF-fed db/db mice. Kaplan-Meyer survival analysis was performed using the log-rank statistics to measure the difference between the survival curves of db/db-HF-S vs db/db-HF-D mice with n=9 per group. Parallel experiments were performed with db/+ mice (db/+-HF-S and db/+-HF-D groups) and no mortality was observed;

FIG. 4 depicts the impact of 17-DMAG on hsp90β1, [Ca²⁺]ₘ, and peroxynitrite generation in the kidney. Western blot analysis was performed to assess hsp90β1 in kidney homogenate (FIG. 4A) and isolated mitochondria (FIG. 4B) of HF-fed db/db mice with 6 animals per group. Linoleic acid-induced [Ca²⁺]ₘ efflux and peroxynitrite generation (FIG. 4C-4F) in kidney mitochondria were measured;

FIG. 5 depicts the effect of 17-DMAG on 3-NT levels in the kidney. Western blot analysis was performed with polyclonal (FIG. 5A) and monoclonal (FIG. 5B) anti-3-NT antibody in kidney homogenates from 6 animals per group. The 3-NT assessment in kidney sections was performed by immunochemistry; and

FIG. 6 depicts the effects of HF and 17-DMAG treatment on bodyweight and blood glucose levels of db/db mice.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular compounds or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise.

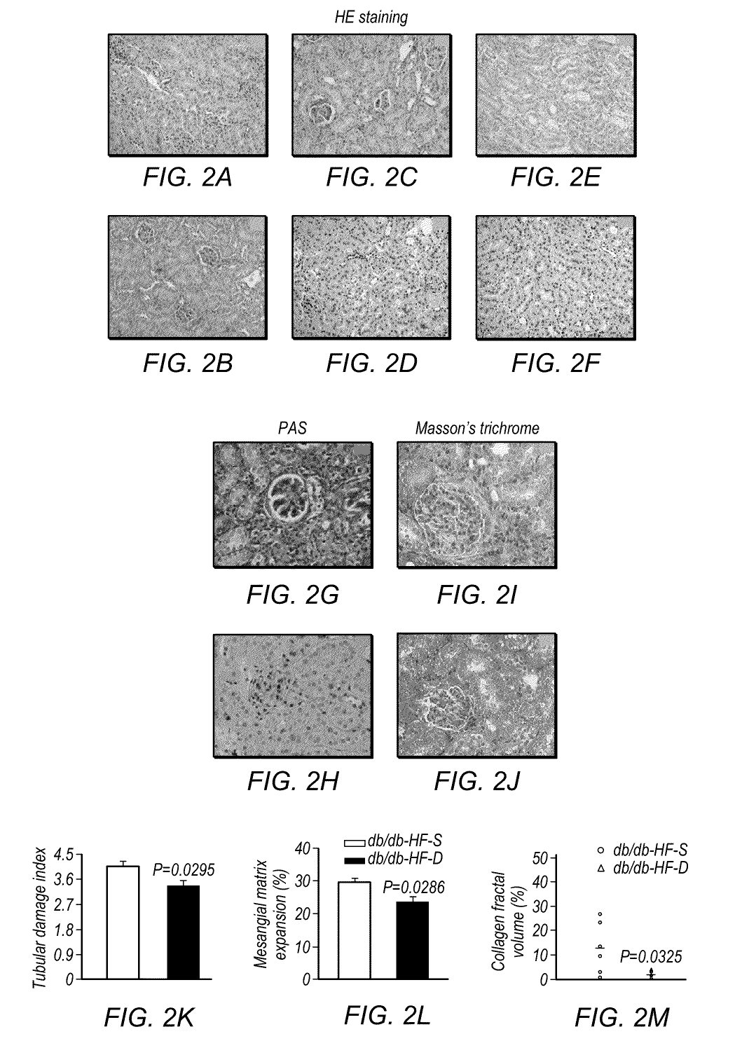

Type 2 diabetes mellitus (“T2DM”) affects a rapidly expanding population worldwide and 80% of T2DM is associated with obesity and a sedentary life style. Among various genetic and environmental factors, imbalanced calorie intake and expenditure play a critical role in the pathogenesis of T2DM. Diabetic nephropathy is a complication that occurs in a subpopulation of T2DM patients after 15-25 years with diabetes. Currently there are no definitive molecular markers to reliably predict its occurrence or its progression to ESRD. ESRD induction in db/db mice by HF feeding indicates that by exaggerating the imbalance between calorie intake and expenditure in T2DM causes progression of diabetic nephropathy to ESRD. The renal histopathology features in HF-fed db/db mice included mesangial matrix expansion, segmental glomerulosclerosis, tubular atrophy and degeneration, tubulointerstitial fibrosis (FIG. 2). In particular, tubular atrophy and interstitial fibrosis have been shown as the key features of ESRD in diabetic patients and correlate with the loss of kidney function. Previously, techniques like unilateral ureteral obstruction and uninephrectomy have been widely used to induce acute progressive renal fibrosis. Following manipulation with these techniques, db/db mice only exhibited tubulointerstitial fibrosis and mesangial matrix accumulation. In contrast, we focused on the impact of metabolic abnormalities on the pathogenesis of ESRD by challenging db/db mice with HF. The resultant model showed severe renal functional insufficiency and most key features of human diabetic ESRD and thus represents a significant advancement in modeling this disease. The natural course of ESRD in T2DM is expected to be much closely mimicked in this db/db-HF model.

Geldanamycin derivatives (e.g., 17-DMAG) exhibit potent anticancer activities against a wide range of malignancies. Its application has been expanded to treat a transgenic mouse model of spinal and bulbar muscular atrophy. It has been found that db/db mice treatment with geldanamycin derivatives during a HF challenge reduces mitochondrial hsp90β1, preserves renal function, and ameliorates damages to glomeruli and tubules, indicating hsp90β1 involvement in development of diabetic ESRD.

Geldanamycin Derivatives have the Structure:

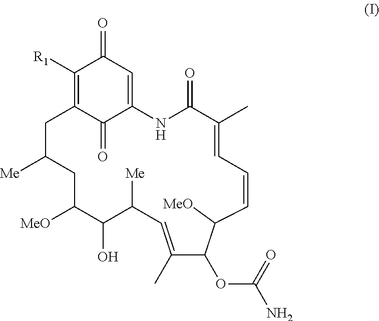

where R₁ is —OR₂ or —NR₃R₄;
where R₂ is hydrogen or C₁-C₆ alkyl;
where R₃ is hydrogen or C₁-C₆ alkyl;
where R₄ is hydrogen, C₁-C₆ alkyl, C₂-C₆ alkenyl or —(CH₂)ₙ—NR₅R₆;
where R₅ is hydrogen or C₁-C₆ alkyl;
where R₆ is hydrogen or C₁-C₆ alkyl; and
where n is 1-6;
or pharmaceutically acceptable salts thereof.

In an embodiment, R₁ is —NR₃R₄; R₃ is hydrogen or C₁-C₆ alkyl; and R₄ is hydrogen, C₁-C₆ alkyl, or C₁-C₆ alkenyl. An exemplary compound is a compound represented by formula (II) (17-(allylamino)-17-demethoxygeldanamycin, 17-AAG).

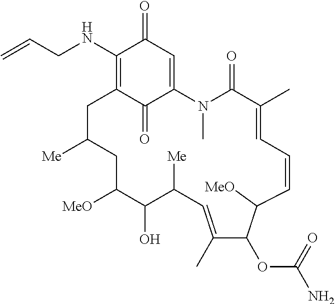 [SMILES: C=CCNC1=C2CC(C)CC(OC)C(O)C(C)/C=C(\C)C(OC(N)=O)C(OC)/C=C\C=C(/C)C(=O)N(C)C(=CC1=O)C2=O]

### 17-(allylamino)-17-demethoxygeldanamycin, 17-AAG (II)

In an embodiment, R₁ is —NR₃R₄; R₃ is hydrogen or C₁-C₆ alkyl; R₄ is —(CH₂)ₙ—NR₅R₆; R₅ is hydrogen or C₁-C₆ alkyl; R₆ is hydrogen or C₁-C₆ alkyl; and n is 1-6. An exemplary compound is a compound represented by formula (III) (17-dimethylaminoethylamino-17-demethoxygeldanamycin, 17-DMAG).

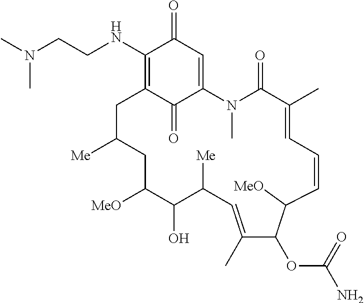 [SMILES: COC1/C=C\C=C(/C)C(=O)N(C)C2=CC(=O)C(NCCN(C)C)=C(CC(C)CC(OC)C(O)C(C)/C=C(\C)C1OC(N)=O)C2=O]

### 17-dimethylaminoethylamino-17-demethoxygeldanamycin (III)

Further information regarding the synthesis and use of geldanamycin derivatives may be found in PCT Publication No. WO 02/079167 entitled “Geldanamycin derivative and method of treating cancer using same” and U.S. Pat. No. 4,261,989 entitled “Geldanamycin derivatives and antitumor drug”, both of which are incorporated herein by reference.

The term “alkyl” as used herein generally refers to a chemical substituent containing the monovalent group CₙH₂ₙ, where n is an integer greater than zero. The term “alkyl” includes branched or unbranched monovalent hydrocarbon radicals. A “C₁-C₆ alkyl” or refers to all alkyl groups containing from 1 to 6 carbon atoms. All possible isomers of an indicated alkyl are also included. Thus, propyl includes isopropyl, butyl includes n-butyl, isobutyl and t-butyl, and so on.

The term “C₂-C₆ alkenyl” as used herein generally refer to any structure or moiety having the at least one unsaturated group C≡C and from 1 to 6 carbon atoms. Examples of compounds having the structure —NR₃R₄ include, but are not limited to nitrogen radicals of: methylamine, ethylamine, propylamine, butylamine, pentylamine, hexylamine, allylamine, β-hydroxyethylamine, β-chloroethylamine, β-glycoxyethylamine, aminobutylamine, cyclopropylamine, cyclopentylamine, cyclohexylamine, dimethylamine, aminoethylamine, β-hydroxyethylamine, and diglycolamine.

In one embodiment, the geldanamycin derivatives described herein may be formulated as a pharmaceutical composition. Any suitable route of administration may be employed for providing a patient with an effective dosage of drugs of the present invention. For example, oral, rectal, topical, parenteral, ocular, intracranial, pulmonary, nasal, and the like may be employed. Dosage forms may include tablets, troches, dispersions, suspensions, solutions, capsules, creams, ointments, oils, emulsions, liposomes, aerosols, and the like. In certain embodiments, it may be advantageous that the compositions described herein be administered orally. In other embodiments, it may be advantageous that the compositions described herein be administered parenterally. In yet other embodiments, it may be advantageous that the compositions described herein be administered locally, at the site of tissue injury.

The pharmaceutical compositions may include those compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well known in the art of pharmacy.

For administration by inhalation, the compositions described herein are conveniently delivered in the form of an aerosol spray presentation from pressurized packs or nebulisers. The compositions may also be delivered as powders which may be formulated and the powder composition may be inhaled with the aid of an insufflation powder inhaler device.

Suitable topical formulations for use in the present embodiments may include transdermal devices, aerosols, creams, ointments, lotions, dusting powders, and the like.

In practical use, compositions can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

The pharmaceutical preparations may be manufactured in a manner which is itself known to one skilled in the art, for example, by means of conventional mixing, granulating, dragee-making, softgel encapsulation, dissolving, extracting, or lyophilizing processes. Thus, pharmaceutical preparations for oral use may be obtained by combining the active compounds with solid and semi-solid excipients and suitable preservatives, and/or co-antioxidants. Optionally, the resulting mixture may be ground and processed. The resulting mixture of granules may be used, after adding suitable auxiliaries, if desired or necessary, to obtain tablets, softgels, lozenges, capsules, or dragee cores.

Suitable excipients may be fillers such as saccharides (e.g., lactose, sucrose, or mannose), sugar alcohols (e.g., mannitol or sorbitol), cellulose preparations and/or calcium phosphates (e.g., tricalcium phosphate or calcium hydrogen phosphate). In addition binders may be used such as starch paste (e.g., maize or corn starch, wheat starch, rice starch, potato starch, gelatin, tragacanth, methyl cellulose, hydroxypropylmethyl-cellulose, sodium carboxymethylcellulose, and/or polyvinyl pyrrolidone). Disintegrating agents may be added (e.g., the above-mentioned starches) as well as carboxymethyl-starch, cross-linked polyvinyl pyrrolidone, agar, or alginic acid or a salt thereof (e.g., sodium alginate). Auxiliaries are, above all, flow-regulating agents and lubricants (e.g., silica, talc, stearic acid or salts thereof, such as magnesium stearate or calcium stearate, and/or polyethylene glycol, or PEG). Dragee cores are provided with suitable coatings, which, if desired, are resistant to gastric juices. Softgelatin capsules (“softgels”) are provided with suitable coatings, which, typically, contain gelatin and/or suitable edible dye(s). Animal component-free and kosher gelatin capsules may be particularly suitable for the embodiments described herein for wide availability of usage and consumption. For this purpose, concentrated saccharide solutions may be used, which may optionally contain gum arabic, talc, polyvinyl pyrrolidone, polyethylene glycol (PEG) and/or titanium dioxide, lacquer solutions and suitable organic solvents or solvent mixtures, including dimethylsulfoxide (DMSO), tetrahydrofuran (THF), acetone, ethanol, or other suitable solvents and co-solvents. In order to produce coatings resistant to gastric juices, solutions of suitable cellulose preparations such as acetylcellulose phthalate or hydroxypropylmethyl-cellulose phthalate, may be used. Dye stuffs or pigments may be added to the tablets or dragee coatings or softgelatin capsules, for example, for identification or in order to characterize combinations of active compound doses, or to disguise the capsule contents for usage in clinical or other studies.

Other pharmaceutical preparations that may be used orally include push-fit capsules made of gelatin, as well as soft, thermally sealed capsules made of gelatin and a plasticizer such as glycerol or sorbitol. The push-fit capsules may contain the active compounds in the form of granules that may be mixed with fillers such as, for example, lactose, binders such as starches, and/or lubricants such as talc or magnesium stearate and, optionally, stabilizers and/or preservatives. In soft capsules, the active compounds may be dissolved or suspended in suitable liquids, such as fatty oils such as rice bran oil or peanut oil or palm oil, or liquid paraffin. In some embodiments, stabilizers and preservatives may be added.

In some embodiments, pulmonary administration of a pharmaceutical composition may be desirable. Pulmonary administration may include, for example, inhalation of aerosolized or nebulized liquid or solid particles of the pharmaceutically active component dispersed in and surrounded by a gas.

Possible pharmaceutical preparations, which may be used rectally or vaginally, include, for example, suppositories, which include a combination of the active compounds with a suppository base. Suitable suppository bases are, for example, natural or synthetic triglycerides, or paraffin hydrocarbons. In addition, it is also possible to use gelatin rectal capsules that consist of a combination of the active compounds with a base. Possible base materials include, for example, liquid triglycerides, polyethylene glycols, or paraffin hydrocarbons.

Suitable formulations for parenteral administration include, but are not limited to, aqueous solutions of the active compounds in water-soluble and/or water dispersible form, for example, water-soluble salts, esters, carbonates, phosphate esters or ethers, sulfates, glycoside ethers, together with spacers and/or linkers. Suspensions of the active compounds as appropriate oily injection suspensions may be administered, particularly suitable for intramuscular injection. Suitable lipophilic solvents, co-solvents (such as DMSO or ethanol), and/or vehicles including fatty oils, for example, rice bran oil or peanut oil and/or palm oil, or synthetic fatty acid esters, for example, ethyl oleate or triglycerides, may be used. Aqueous injection suspensions may contain substances that increase the viscosity of the suspension including, for example, sodium carboxymethyl cellulose, sorbitol, dextran, and/or cyclodextrins. Cyclodextrins (e.g., β-cyclodextrin) may be used specifically to increase the water solubility for parenteral injection of the compound. Liposomal formulations, in admixture with, for example, egg yolk phosphotidylcholine (E-PC), may be made for injection. Optionally, the suspension may contain stabilizers, for example, antioxidants such as BHT, and/or preservatives, such as benzyl alcohol.

The compositions of this invention can be administered in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. They can be administered alone, but generally will be administered with a pharmaceutical carrier selected on the basis of the chosen route of administration and standard pharmaceutical practice.

The dosage regimen for the compounds will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian may determine and prescribe the effective amount of the drug required to prevent, counter, or arrest the progress or the development prostate cancer in a subject. The pharmaceutical compositions may be administered in a single daily dose, or the total daily dosage may be administered in divided doses of two, three, or four or more times daily.

The pharmaceutical compositions described herein may further be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen.

The compounds are typically administered in admixture with suitable pharmaceutical diluents, excipients, or carriers (collectively referred to herein as “pharmacologically inert carriers”) suitably selected with respect to the intended form of administration, that is, oral tablets, capsules, elixirs, syrups and the like, and consistent with conventional pharmaceutical practices.

For instance, for oral administration in the form of a tablet or capsule, the pharmacologically active component may be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical carriers are described in Remington's Pharmaceutical Sciences, Mack Publishing Company, a standard reference text in this field.

In an embodiment, the pharmaceutical composition may be administered to the patient systemically. The term systemic as used herein includes subcutaneous injection; intravenous, intramuscular, intraestemal injection; infusion; inhalation, transdermal administration, oral administration; and intra-operative instillation.

One systemic method involves an aerosol suspension of respirable particles comprising the active compound, which the subject inhales. The active compound would be absorbed into the bloodstream via the lungs, and subsequently contact the lacrimal glands in a pharmaceutically effective amount. The respirable particles may be liquid or solid, with a particle size sufficiently small to pass through the mouth and larynx upon inhalation; in general, particles ranging from about 1 to 10 microns, but more preferably 1-5 microns, in size are considered respirable.

Another method of systemically administering the active compounds involves administering a liquid/liquid suspension in the form of eye drops or eye wash or nasal drops of a liquid formulation, or a nasal spray of respirable particles that the subject inhales. Liquid pharmaceutical compositions of the active compound for producing a nasal spray or nasal or eye drops may be prepared by combining the active compound with a suitable vehicle, such as sterile pyrogen free water or sterile saline by techniques known to those skilled in the art.

The active compounds may also be systemically administered through absorption by the skin using transdermal patches or pads. The active compounds are absorbed into the bloodstream through the skin. Plasma concentration of the active compounds can be controlled by using patches containing different concentrations of active compounds.

Other methods of systemic administration of the active compound involves oral administration, in which pharmaceutical compositions containing active compounds are in the form of tablets, lozenges, aqueous or oily suspensions, viscous gels, chewable gums, dispersible powders or granules, emulsion, hard or soft capsules, or syrups or elixirs. Additional means of systemic administration of the active compound to the subject may involve a suppository form of the active compound, such that a therapeutically effective amount of the compound reaches the eyes via systemic absorption and circulation.

Further means of systemic administration of the active compound involve direct intra-operative instillation of a gel, cream, or liquid suspension form of a therapeutically effective amount of the active compound.

For topical application, the solution containing the active compound may contain a physiologically compatible vehicle, as those skilled in the art can select, using conventional criteria. The vehicles may be selected from the known pharmaceutical vehicles which include, but are not limited to, saline solution, water polyethers such as polyethylene glycol, polyvinyls such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate and salts such as sodium chloride and potassium chloride.

For systemic administration such as injection and infusion, the pharmaceutical formulation is prepared in a sterile medium. The active ingredient, depending on the vehicle and concentration used, can either be suspended or dissolved in the vehicle. Adjuvants such as local anaesthetics, preservatives and buffering agents can also be dissolved in the vehicle. The sterile injectable preparation may be a sterile injectable solution or suspension in a non-toxic acceptable diluent or solvent. Among the acceptable vehicles and solvents that may be employed are sterile water, saline solution, or Ringer's solution.

In practical use, the geldanamycin derivatives used can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Animals

Male db/db (BKS.Cg-m+/+Lepr^{db}/J), age and sex matched db/+ mice were acquired from Jackson Laboratories at age of 10 weeks and housed 4/cage or less. Animals were habitat for 2 weeks in a temperature- and humidity-controlled facility with a 12:12-h light-dark cycle, fed ad libitum with RD (7012 Teklad LM-485, Harlan Laboratories) and had free access to water.

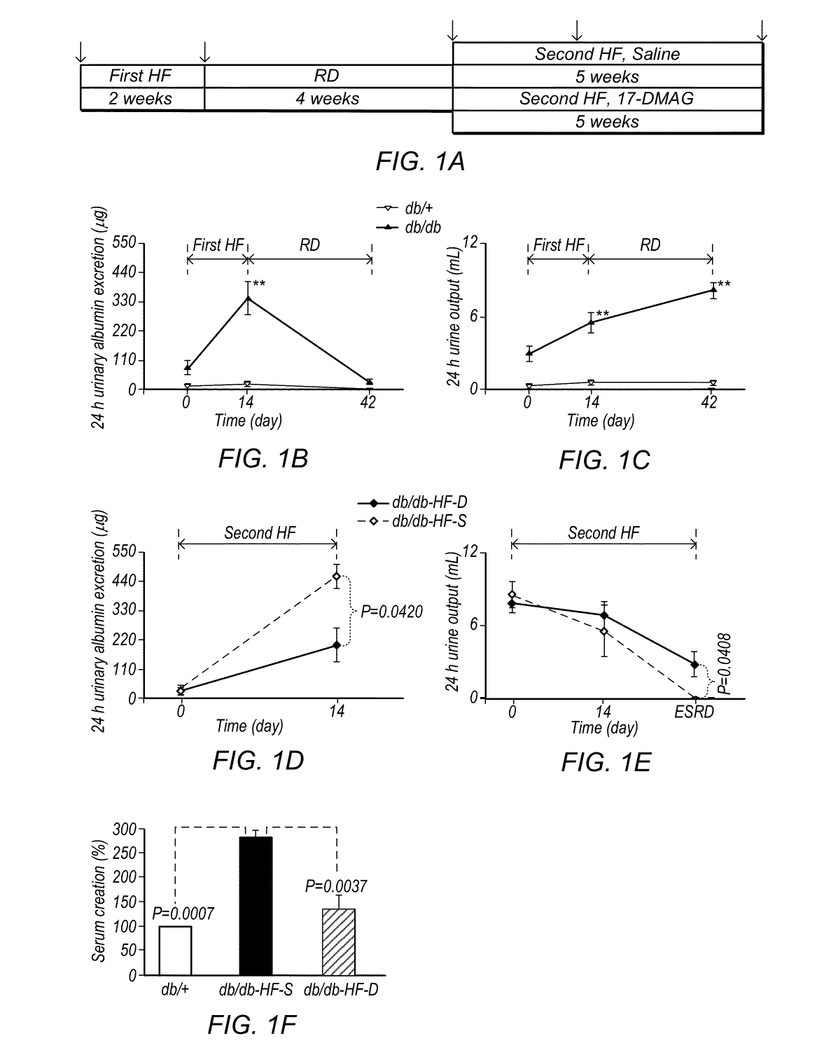

The mice were challenged with HF (60.3% calories from fat, TD.06414, Harlan Laboratories) starting at 12 weeks of age as depicted in FIG. 1A. The HF challenge was divided into two phases: the first phase lasted 2 weeks followed by a 4-week RD feeding. The second phase of HF feeding followed the 4-week RD interval and continued to the end of the experiments (5 weeks). At the beginning of the second HF feeding, db/db mice were randomly divided into 2 groups and intraperitoneally injected with either Saline (vehicle, db/db-HF-S) or 17-DMAG (6.5 μg/Kg bodyweight, db/db-HF-D). The animal protocols were approved by the Institutional Animal Care and Use Committee (0707-001), UT Health Science Center at San Antonio.

### Assessment of 24-h Urine Output and Urinary Albumin

Urine collection and other physical parameters were measured following the schedule in FIG. 1A. 24 h urine samples were collected from individual mice and urinary albumin concentration was determined using a murine albumin enzyme-linked immunosorbent assay (Albuwell M Kit; Exocell, Philadelphia, Pa.). Serum creatinine concentrations were determined by the Creatinine Companion kit (Exocell).

### Histopathology

Formalin-fixed, paraffin-embedded kidney sections were stained with haematoxylin and eosin (HE), periodic acid-Schiff (PAS), or Masson's trichrome and analyzed to evaluate kidney damages in a blinded manner. The area of glomerular PAS staining was measured by image analysis using Image-Pro Plus 4.5 (Media Cybernetics, Silverspring, Md.). A semi-quantitative assessment was performed in HE stained slides to evaluate the extent of tubular damage and graded from 1 to 5 as follows: 1: vacuolation of cytoplasm in <20% of tubules; 2: vacuoles in 20% to 40% of tubules; 3: vacuoles in 40% to 60% tubules with minimal distortion of tubular structures; 4: vacuoles in 60% to 80% of tubules with large and marked distortion of tubular profiles, pyknotic nuclei, patches of tubular atrophy and tubular degeneration; and 5: >80% of tubules with severe vacuolation, or tubular atrophy and degeneration. The fractal collagen volume was assessed by point counter grid using ImageJ (NIH) program to quantify the blue stain in the trichrome-stained sections.

### Measurement of [Ca²⁺]ₘ and Peroxynitrite in Mitochondria.

Kidney mitochondria were prepared from db/+ or db/db mice and LA-induced [Ca²⁺]ₘ and peroxynitrite were assessed as previously described.

Assessment of hsp90β1 and 3-NT

Western blot or immunochemistry was performed with polyclonal (pAb) and/or monoclonal (mAb) primary antibodies to detect hsp90β1 and 3-NT levels as previously described. The primary antibodies used in western blots were as follows: mAb anti-3-NT (1:1000, clone 1A6, Millipore); rabbit pAb anti-3-NT (1:1000, Millipore); pAb anti-GRP94 (1:1000, Santa Cruz Biotechnology), mAb anti-Complex 1 (1:2000) and mAb anti-α-tubulin (1:1000). For the immunochemistry staining with mouse antibody, a specific procedure was performed as previously described to eliminate the direct interaction between antigen and secondary antibody.

### Statistical Analysis

Results presented as mean±S.E and Student's t-test was used to evaluate the differences between two groups. Kaplan-Meyer survival analysis was performed using the log-rank statistic to test for a significant difference among the survival curves. Differences were considered statistically significant at P<0.05.

### 17-DMAG Ameliorates HF-Induced ESRD

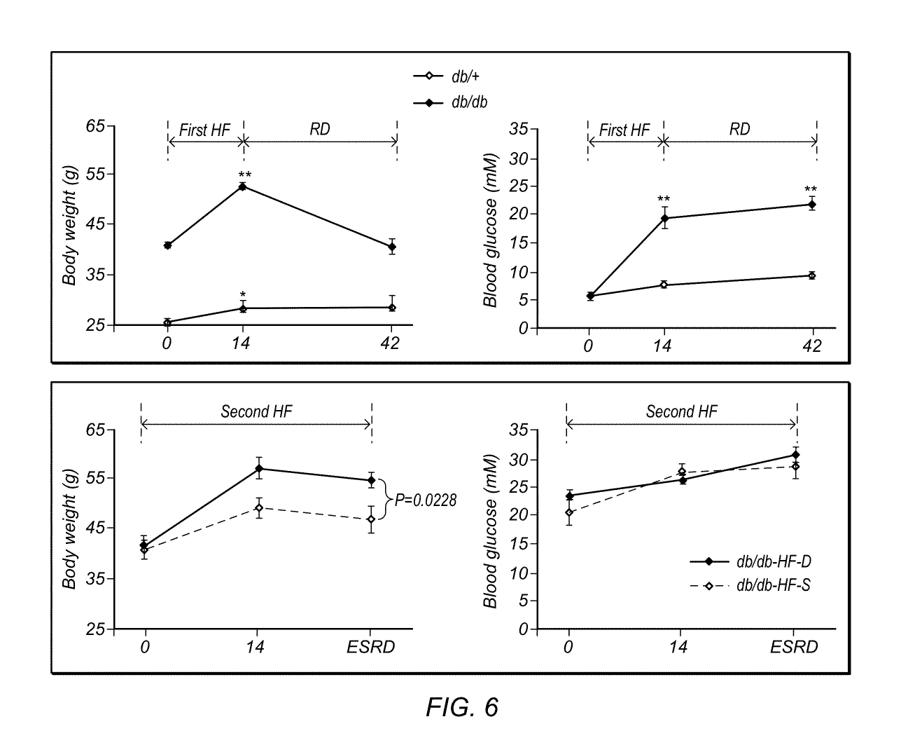

Challenge of db/db mice with HF was divided into two phases with a four-week interval with a regular diet (RD) as illustrated in FIG. 1A. Following first HF feeding, all db/db mice showed dramatic increases in urinary albumin excretion and urine output (FIGS. 1B and C), rapid gain in bodyweight, and elevated blood glucose levels (FIG. 6). The urinary albumin excretion (FIG. 1B) and bodyweight (FIG. 6) were fully reversed to the pre-treatment levels four weeks after switching to RD. However, increased urine output and blood glucose levels persisted even after returning to RD. In parallel control experiments, two-week HF challenge had no significant effects on urinary albumin excretion and urine output in db/+mice (FIGS. 1B and C).

To assess the impact of 17-DMAG, a geldanamycin derivative that inhibits the chaperone activity of hsp90, on renal functions and ESRD development, mice were either injected intraperitoneally with saline (vehicle, db/db-HF-S) or 17-DMAG (db/db-HF-D, 6.5 μg/kg bodyweight) daily at the beginning of the second HF challenge. The dose of 17-DMAG was based on our previous studies showing 17-DMAG exerting maximal inhibition on PIMCE but did not produce apparent toxicity to cells. The robust albuminuria observed in the first HF feeding was fully reproduced in db/db-HF-S group and was significantly diminished by 17-DMAG in db/db-HF-D group (FIG. 1D). It has been demonstrated that the dramatically reduced 24 h urine volume is closely correlated with loss of estimated glomerular filtration rate (GFR) and ESRD development providing a reliably index of residual renal function. Starting from the 17^{th} day of the second HF feeding, we observed a dramatic loss of urine output or anuria in db/db-HF-S group, indicating the development of ESRD, while 17-DMAG treatment significantly preserved 24 h urine output in db/db-HF-D group (FIG. 1 E), indicating preservation of renal function by 17-DMAG.

Because urinary albumin excretion measurement was not practical following ESRD development due to oliguria and anuria, serum creatinine was analyzed to assess kidney function. Serum creatinine of db/db-HF-S group at the time of ESRD was 283% higher than that of db/+ mice, indicating failure of the kidney to clear creatinine Furthermore, db/db-HF-D group had significantly lowered serum creatinine compared to the db/db-HF-S group (FIG. 1F) and no significant difference was found between db/db-HF-D and db/+ groups, indicating that 17-DMAG restored mice to normal creatinine clearance. These data provided evidence that HF challenge resulted in renal failure in db/db mice and 17-DMAG treatment effectively preserved renal function.

### 17-DMAG Mitigates HF-Induced Damages of Glomeruli and Tubules

Incremental tubulointerstitial fibrosis development over time is closely correlated with loss of renal function or ESRD development. To assess the histopathology features of glomeruli and tubules in HF-fed db/db mice that developed ESRD, the kidneys were collected at the time of renal failure (as indicated by oliguria and anuria) or at the end of the experiments (day 34 of HF feeding) and analyzed by histopathological stains. Hematoxylin-eosin (HE) staining at low power (100×) indicated segmental glomerulosclerosis in the db/db-HF-S group (FIG. 2C) and the damage was largely prevented by 17-DMAG in db/db-HF-D group (FIG. 2D). Furthermore, patches of tubular atrophy and degeneration were found in the tubules of db/db-HF-S group (FIG. 2E) while 17-DMAG treatment provided dramatic protection against these damages (FIG. 2F). However, tubular vacuolation was evidenced in both db/db-HF-S and db/db-HF-D groups. In parallel control experiments, HF-fed db/+ mice treated with saline (FIG. 2A) or 17-DMAG (FIG. 2B) did not exhibit any glomerular and tubular abnormalities.

Inspection of PAS stained sections under higher magnification (200×) confirmed segmental glomerulosclerosis and mesangial matrix expansion in db/db-HF-S group (FIG. 2G); 17-DMAG treatment amiiorated these glomerular lesions (FIG. 2H). A strongly PAS-stained tubulointerstitium suggested tubulointerstitial fibrosis in db/db-HF-S group (FIG. 2G), which was largely prevented in the db/db-HF-D group (FIG. 2H). Tubulointerstitial fibrosis in db/db-HF-S group and the impact of 17-DMAG treatment were further demonstrated by Masson's trichrome staining The results showed marked accumulation of tubulointerstitial collagen in db/db-HF-S group (FIG. 2I); 17-DMAG treatment effectively reduced this abnormality (FIG. 2J). Quantification of these stains with morphometric measurements confirmed significant alleviation of tubular damage (FIG. 2K), mesangial matrix expansion (FIG. 2L), and collagen accumulation (FIG. 2M) by 17-DMAG treatment.

17-DMAG Improves the Survival of db/db Mice with ESRD

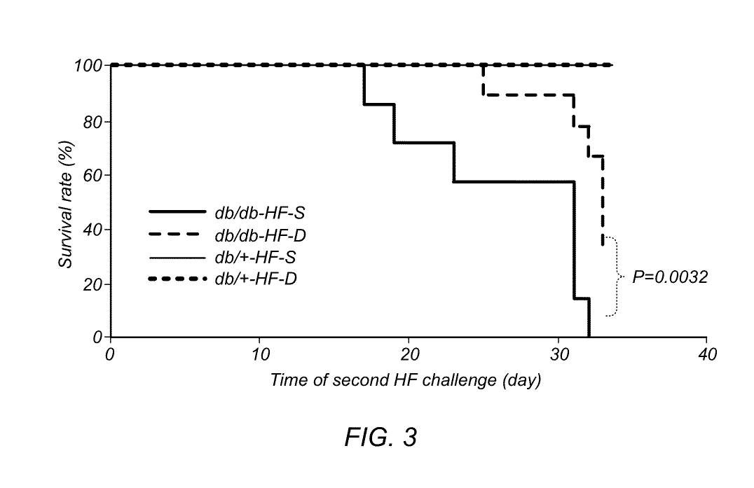

Patients with ESRD (i.e. loss of >80% GFR) need dialysis or replacement therapy to survive. In mice or other animal models, ESRD is expected to cause high mortalities without dialysis or kidney transplantation. As demonstrated in FIG. 3, the earliest death in db/db-HF-S mice due to ESRD was observed on the 17^{th} day of second HF challenge and a complete mortality was observed within 33 days. Treatment with 17-DMAG resulted in a significantly improved survival rate in db/db-HF-D group. HF feeding did not cause ESRD or mortality in db/+ mice regardless of the treatments.

17-DMAG Reduces Kidney Mitochondrial hsp90β1 Levels and Inhibits LA-Induced [Ca²⁺]ₘ Efflux:

We investigated the potential mechanisms underlying the ESRD development in HF-challenged db/db mice. Our previous in vitro studies indicated that NEFA, particularly PUFA interacted with hsp90β1 to induce PIMCE and peroxynitrite generation. Peroxynitrite attacks proteins and converts tyrosine residues to 3-NT that leads to nitrosative injuries to cells and tissues. Increased 3-NT in the plasma and kidney of diabetic patients has been observed and could be a potential risk factor for diabetic ESRD. Here we focused on hsp90β1, [Ca²⁺]ₘ and peroxynitrite generation in the kidney of HF challenged db/db mice to investigate contribution to ESRD.

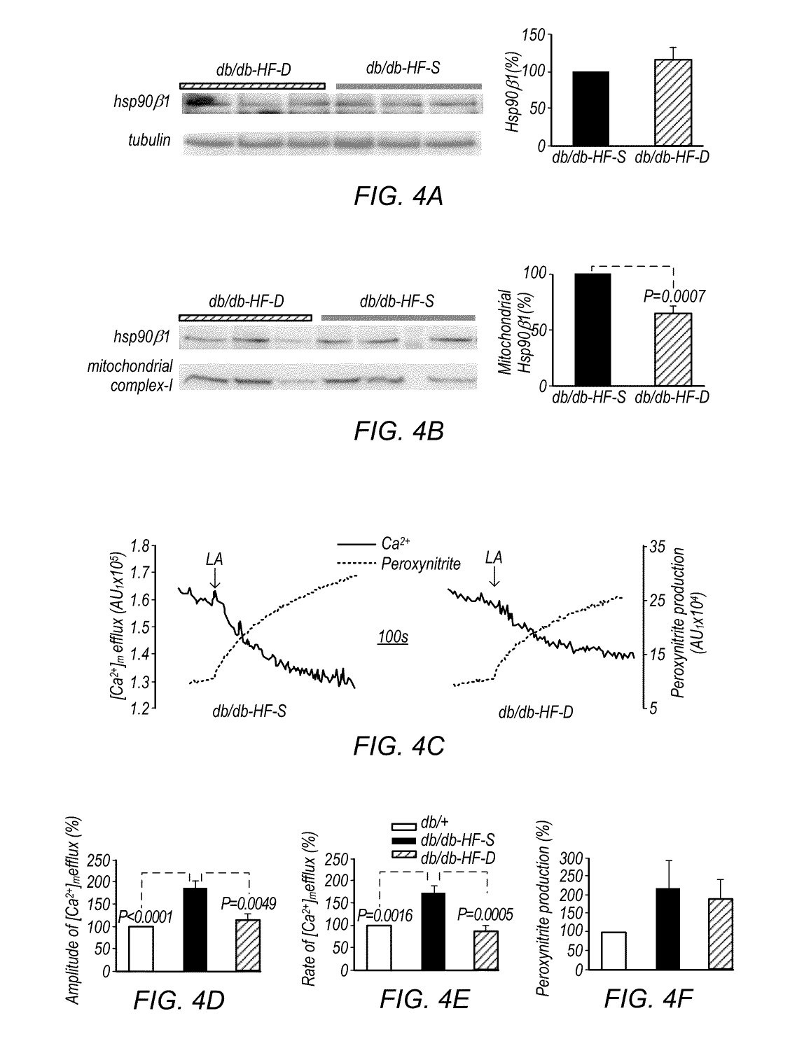

It has been demonstrated that prolonged treatment of cells with geldanamycin and 17-DMAG caused degradation and downregulation of hsp90β1. We measured the effect of daily 17-DMAG injection on hsp90β1 levels in the kidney of db/db-HF mice. The results demonstrated similar hsp90β1 levels in the kidney homogenates between db/db-HF-S and db/db-HF-D groups (FIG. 4A). Interestingly, the hsp90β1 levels in isolated kidney mitochondria of db/db-HF-D group were significantly reduced compared to db/db-HF-S group (FIG. 4B).

In primary human mesangial cells, mitochondria-localized hsp90β1 was involved in Ca²⁺ efflux from mitochondria, peroxynitrite formation and enhanced nitrotyrosylation of cellular proteins in response to LA. As shown in FIG. 4C-E, LA-induced [Ca²⁺]ₘ efflux was significantly diminished in the kidney mitochondria of db/db-HF-D group compared to db/db-HF-S group consistent with our in vitro studies. Both the rate and the amplitude of LA-induced [Ca²⁺]ₘ signal in the db/db-HF-S group were significantly higher than those of db/+ mice; 17-DMAG treatment effectively attenuated the LA-induced [Ca²⁺]ₘ responses in db/db-HF-D group (FIGS. 4D and 4E). There were no significant differences in the rate and amplitude of LA-induced [Ca²⁺]ₘ signal between db/db-HF-D and db/+ groups, indicating that 17-DMAG restored LA-induced [Ca²⁺]ₘ response to normal levels. The coupled LA-induced peroxynitrite generation was also attenuated by 17-DMAG in db/db-HF-D group even though the results did not reach statistical significance (FIG. 4F). These data demonstrate that in HF-fed db/db mice, 17-DMAG reduces mitochondrial hsp90β1 levels and LA-induced [Ca²⁺]ₘ efflux, which may preserve Ca²⁺-dependent mitochondrial functions and inhibit superoxide production in the kidney.

### 17-DMAG Reduces 3-NT Levels in the Kidney

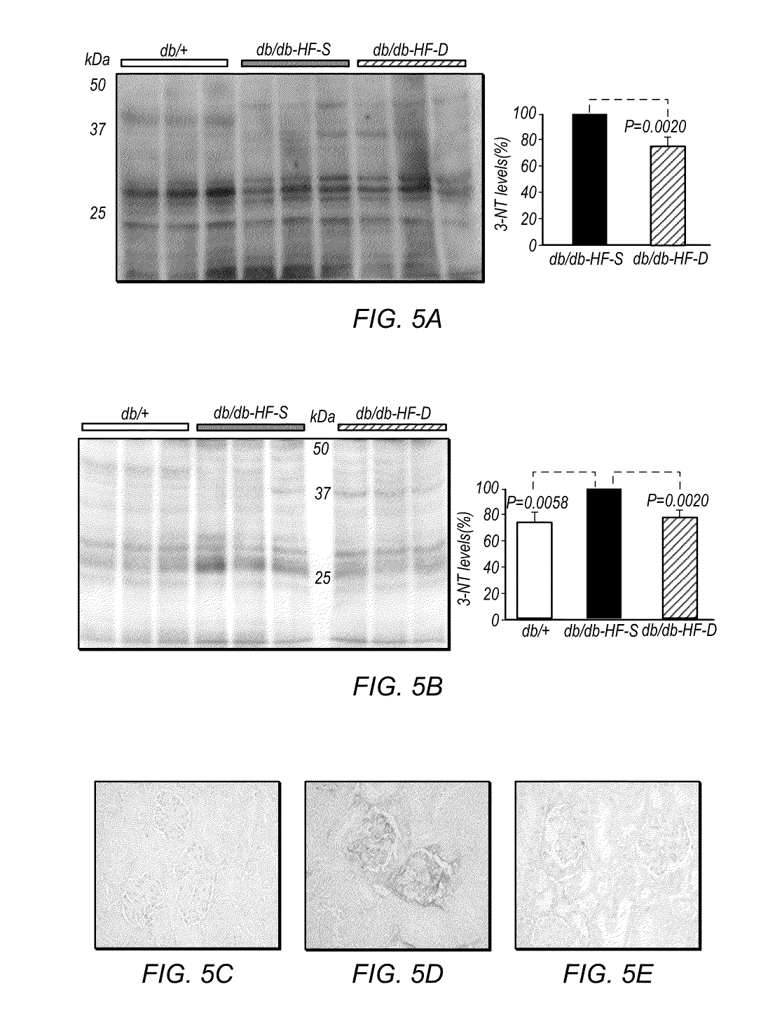

The 3-NT levels in kidney were assessed by western blot (FIGS. 5A and B) and immunohistochemistry (FIG. 5C-E). As demonstrated in FIG. 5A, multiple protein bands containing 3-NT in db/+, db/db-HF-S, and db/db-HF-D groups were detected with a polyclonal anti-3-NT antibody. Quantification of the bands with MW<50kDa indicated significant 3-NT level reduction in the db/db-HF-D group compared to the db/db-HF-S group. However, the quantified results of db/db mice cannot be directly compared to those of db/+ mice because 3-NT was detected at different protein bands. We next probed 3-NT with a monoclonal antibody (FIG. 5B) and the detected protein bands were in a comparable manner. 3-NT levels in proteins <50 kDa of db/db-HF-S group were significantly higher compared to db/+ mice; 17-DMAG treatment significantly lowered the 3-NT levels (FIG. 5B) in db/db-HF-D group, indicating protective effect of 17-DMAG against nitrosative injury. Analysis with immunohistochemistry confirmed high 3-NT levels in db/db-HF-S group, and 17-DMAG treatment effectively reduced 3-NT in db/db-HF-D group (FIG. 5C-E).

Effects of HF and 17-DMAG Treatment on Bodyweight and Blood Glucose Levels of db/db Mice

HF caused rapid gains in bodyweight that was reversed to the baseline values measured prior to HF feeding following four weeks feeding with RD. The elevation of blood glucose after first HF seemed irreversible even after four weeks on RD feeding. The bodyweight gain was significantly potentiated in db/db-HF-D group compared to db/db-HF-S group (supplemental FIG. 6). The persistently elevated blood glucose level that were not reversed during the four week interval of RD did not show further significant increases in the second HF challenge (supplemental FIG. 6, *P<0.05, **P<0.001, compared to baseline values assessed on day 0 from 6-12 animals per group).

As a chaperone protein, hsp90β1 is abundantly expressed in cells and tissues and is widely distributed in most subcellular organelles, such as plasma membrane, cytosol, endoplasmic reticulum (ER), mitochondrion, and nucleus. The mitochondrial hsp90β1 in tumor cells forms a chaperone network and serves as an important regulator of mitochondrial integrity and apoptosis. Compared to healthy controls, increased hsp90β levels have been observed in muscles of T2DM patients. Hsp90 were detected in outer medulla and glomeruli of rat kidney, but no significant alterations were observed in type 1 diabetic rats. Our previous in vitro studies have indicated that mitochondrial hsp90β1 is involved in regulation of cytosolic Ca²⁻ and [Ca²⁺]ₘ homeostasis. A major role of [Ca²⁺]ₘ is to stimulate oxidative phosphorylation by activation of multiple dehydrogenases and ATP synthesis. [Ca²⁺]ₘ inhibits the generation of reactive oxygen species (ROS) from complexes I and III under normal conditions whereas its overload promotes ROS generation and apoptosis. The enhanced [Ca²⁻]ₘ efflux in HF-fed db/db mice diminishes the inhibitory effect of [Ca²⁺]ₘ on ROS production and thus leads to overproduction of superoxide and peroxynitrite. In our db/db-HF model, elevated NEFA, particularly PUFA (such as LA) due to excessive fat intake, presumably interact with hsp90β1 to deplete [Ca²]ₘ and overproduce peroxynitrite which exaggerates kidney nitrosative injuries and drives development of diabetic ESRD. The enhanced [Ca²⁺]ₘ efflux and 3-NT levels in the kidney of db/db-HF mice and the protective effects of geldanamycin derivative 17-DMAG (FIGS. 4 and 5) provide strong support in favor of this scenario. The significant elevation of plasma n-6 PUFA (LA and AA) and kidney 3-NT levels in T2DM patients indicate that a similar scenario may also occur in humans.

In summary, the establishment of diabetic ESRD in db/db mice with the induction of severe renal insufficiency and interstitial fibrosis represents a major breakthrough for diabetic ESRD research and provides a suitable animal model for investigation of human diabetic ESRD. Our technique focuses on excessive lipid and calorie intake in a well-defined T2DM model and the process closely mimics the dramatic metabolic abnormalities occurred in patients with T2DM and obesity. Our results provide support that HF causes diabetic nephropathy progression to ESRD by disrupting [Ca²⁺]ₘ homeostasis and overproducing peroxynitrite, resulting in enhanced 3-NT and nitrosative injury in the kidney. 17-DMAG downregulates mitochondrial hsp90β1 level and prevents the depletion of [Ca²⁺]ₘ as well as peroxynitrite overproduction, which contribute to diminished nitrosative injury and preserved kidney function as well as lowered animal mortality from ESRD.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method of alleviating end-stage renal disease (ESRD), comprising administering an effective amount of a compound having the structure (I): 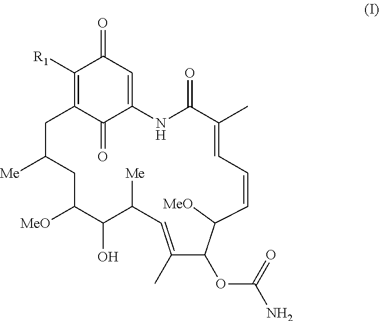
where R₁ is —OR₂ or —NR₃R₄;
where R₂ is hydrogen or C₁-C₆ alkyl;
where R₃ is hydrogen or C₁-C₆ alkyl;
where R₄ is hydrogen, C₁-C₆ alkyl, C₂-C₆ alkenyl or —(CH₂)ₙ—NR₅R₆;
where R₅ is hydrogen or C₁-C₆ alkyl;
where R₆ is hydrogen or C₁-C₆ alkyl; and
where n is 1-6;
or pharmaceutically acceptable salts thereof.

2. The method of claim 1, wherein R₁ is —NR₃R₄; R₃ is hydrogen or C₁-C₆ alkyl; and R₄ is hydrogen, C₁-C₆ alkyl, or C₁-C₆ alkenyl.

3. The method of claim 1, wherein the compound is 17-(allylamino)-17-demethoxygeldanamycin (17-AAG).

4. The method of claim 1, wherein R₁ is —NR₃R₄; R₃ is hydrogen or C₁-C₆ alkyl; R₄ is —(CH₂)ₙ—NR₅R₆; R₅ is hydrogen or C₁-C₆ alkyl; R₆ is hydrogen or C₁-C₆ alkyl; and n is 1-6.

5. The method of claim 1, wherein the compound is 17-(dimethylaminoethylamino)-17-demethoxygeldanamycin (17-DMAG).

6. A method of improving the pathologic features associated with diabetic nephropathy (kidney damage) including renal hypertrophy, glomerular enlargement, albuminuria and mesangial expansion, comprising administering an effective amount of a compound having the structure (I): 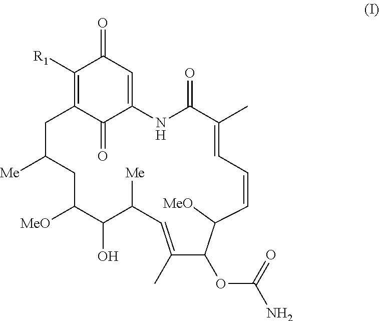
where R₁ is OR₂ or —NR₃R₄;
where R₂ is hydrogen or C₁-C₆ alkyl;
where R₃ is hydrogen or C₁-C₆ alkyl;
where R₄ is hydrogen, C₁-C₆ alkyl, C₂-C₆ alkenyl or —(CH₂)ₙ—NR₅R₆;
where R₅ is hydrogen or C₁-C₆ alkyl;
where R₆ is hydrogen or C₁-C₆ alkyl; and
where n is 1-6;
or pharmaceutically acceptable salts thereof.

7. The method of claim 6, wherein R₁ is —NR₃R₄; R₃ is hydrogen or C₁-C₆ alkyl; and R₄ is hydrogen, C₁-C₆ alkyl, or C₁-C₆ alkenyl.

8. The method of claim 6, wherein the compound is 17-(allylamino)-17-demethoxygeldanamycin (17-AAG).

9. The method of claim 6, wherein R₁ is —NR₃R₄; R₃ is hydrogen or C₁-C₆ alkyl; R₄ is —(CH₂)ₙ—NR₅R₆; R₅ is hydrogen or C₁-C₆ alkyl; R₆ is hydrogen or C₁-C₆ alkyl; and n is 1-6.

10. The method of claim 6, wherein the compound is 17-(dimethylaminoethylamino)-17-demethoxygeldanamycin (17-DMAG).

11. A method of attenuating the symptoms of diabetic nephropathy in patients diagnosed with type 2 diabetes mellitus (T2DM), comprising administering an effective amount of a compound having the structure (I): 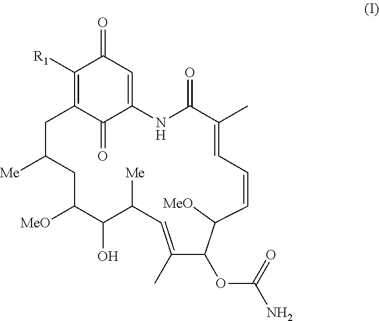
where R₁ is —OR₂ or —NR₃R₄;
where R₂ is hydrogen or C₁-C₆ alkyl;
where R₃ is hydrogen or C₁-C₆ alkyl;
where R₄ is hydrogen, C₁-C₆ alkyl, C₂-C₆ alkenyl or —(CH₂)ₙ—NR₅R₆;
where R₅ is hydrogen or C₁-C₆ alkyl;
where R₆ is hydrogen or C₁-C₆ alkyl; and
where n is 1-6;
or pharmaceutically acceptable salts thereof.

12. The method of claim 11, wherein R₁ is —NR₃R₄; R₃ is hydrogen or C₁-C₆ alkyl; and R₄ is hydrogen, C₁-C₆ alkyl, or C₁-C₆ alkenyl.

13. The method of claim 11, wherein the compound is 17-(allylamino)-17-demethoxygeldanamycin (17-AAG).

14. The method of claim 11, wherein R₁ is —NR₃R₄; R₃ is hydrogen or C₁-C₆ alkyl; R₄ is —(CH₂)ₙ—NR₅R₆; R₅ is hydrogen or C₁-C₆ alkyl; R₆ is hydrogen or C₁-C₆ alkyl; and n is 1-6.

15. The method of claim 11, wherein the compound is 17-(dimethylaminoethylamino)-17-demethoxygeldanamycin (17-DMAG).
